(19) United States
(12) Patent Application Publication
(10) Pub. No.: US 2025/0073149 A1
(43) Pub. Date: Mar. 6, 2025

(54) COMPOSITIONS CONTAINING AT LEAST ONE BIOSURFACTANT AND AT LEAST ONE SULFONIC OR SULFINIC ACID DERIVATIVE

(71) Applicant: Evonik Operations Gmbh, Essen (DE)

(72) Inventors: Jakob Müller, Haltern am See (DE); Stefan Julian Liebig, Duesseldorf (DE); Christoph Tölle, Duisburg (DE); Hans Henning Wenk, Muelheim an der Ruhr (DE)

(73) Assignee: Evonik Operations GmbH, Essen (DE)

(21) Appl. No.: 18/558,976

(22) PCT Filed: Apr. 28, 2022
(86) PCT No.: PCT/EP2022/061335

§ 371 Date: Nov. 3, 2023

(30) Foreign Application Priority Data
May 5, 2021 (EP) 21172184.0

(51) Int. Cl. A61K 8/46; A61K 8/60; A61Q 19/10

(52) U.S. Cl. CPC A61K 8/466; A61K 8/602; A61Q 19/10; A61K 2800/75

(57) ABSTRACT

Compositions containing at least one biosurfactant and at least one sulfonic or sulfinic acid derivative can be used in formulations

### FIELD OF THE INVENTION

The invention relates to compositions containing at least one biosurfactant and at least one sulfonic or sulfinic acid derivative and their uses.

### PRIOR ART

In the field of cosmetics or household care applications, colour and odour of formulations play an important role in the consumer perception. Like many other natural products biosurfactants are known to have a characteristic colour and odour, which needs to be reduced, so that the biosurfactants as raw material fulfil the market's needs.

WO2016115048 describes process steps of deodorising and decolourising rhamnolipid compositions. One disadvantage of the described invention is the inclusion of an additional process step in the production of rhamnolipids.

EP2821495 discloses a method for solving the problem to sophorolipid-containing compositions having a reduced smell by providing a high-purity acid-form sophorolipid.

WO2013129667 addresses the problem of sophorolipids suffering of irritative odour due to fermentation by-products generated in the fermentation generation processes.

WO2018108925 discloses sulfonic acid salt compositions useful for colour stabilization of non-living organic matter such as from lacquers, paints, powder-coatings, and polymers in particular water-absorbent polymers. The writ states, that sulphurous reducing agents in general are of unpleasant odour, which is unwanted in the final polymer products. It is disclosed, that the disodium salt of the used sulfonic acid has a very well noticeable odour. Salts of multivalent metals as zinc, aluminium and magnesium of 2-xydroxy-2-sulfonatoacetate are disclosed to bear a reduced odour.

WO2004084962 discloses a colour-stable superabsorbent polymer having long-term colour stability, which is prepared using a sulfinic acid derivative, like 2-hydroxy-2-sulfinatoacetic acid, as the reducing agent in the polymerization initiator system.

It is an object of the invention to provide compositions containing biosurfactants with an improved odour profile.

### DESCRIPTION OF THE INVENTION

It has been found that the addition of small amounts of sulfonic or sulfinic acid derivatives have a positive effect on the odour profile of different biosurfactants.

This is surprising, as usually, as e.g. described in WO2018108925, the odour of sulfonic or sulfinic acid derivatives is subject to optimization, the combination with different biosurfactants leads to an overall positive odour signature.

The present invention therefore provides
compositions containing
   A) at least one biosurfactant
   B) at least one sulfonic or sulfinic acid derivative of general formula (I)

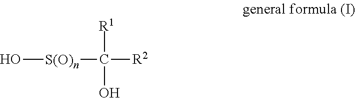

with
n=1 or 2,
R¹ selected from H and C₁-C₆ alkyl,
R² selected from COOH, SO₃H and CH(OH)SOₘ—OH with m=1 or 2, preferably m=n,
or any salt thereof.

One advantage of the present invention is that the compositions according to the instant invention have a positive odour profile.

Another advantage of the present invention is that the compositions according to the instant invention have an improved colour stability. Colour change reactions could come from Maillard reaction, which is a chemical reaction between proteins or amino acids and sugars. Both reaction partners, proteins and sugars, are typically contained in small amounts in a fermentative produced biosurfactant like Rhamnolipids, Sophorolipids or Rubiwettin. These small concentrations are sufficient to have a colour change from light to dark.

A further advantage is that the compositions according to the instant invention have improved cleaning properties of surfaces.

Another advantage is that the compositions according to the instant invention has excellent foaming properties.

A further advantage is that the compositions according to the instant invention is their reduced irritation for human skin.

Another advantage is that the compositions according to the instant invention is their mildness and good physiological compatibility, in particular characterized by a high value in the red blood cell (RBC) test.

A further advantage is that the compositions according to the instant invention is their good skin feel during and after washing.

Another advantage is that the compositions according to the instant invention is that they leave behind a smooth and soft skin feel after washing.

Another advantage is that the compositions according to the instant invention is their outstanding microbial stability.

Another advantage is that the compositions according to the instant invention have an improved ability to dissolve oils and fats.

Within the context of the present invention, biosurfactants are understood as meaning all glycolipids produced by fermentation. The term “biosurfactant” also covers glycolipids that are chemically or enzymatically modified after fermentation, as long as structurally a glycolipid remains. Raw materials for producing the biosurfactants that can be used are carbohydrates, in particular sugars such as e.g. glucose and/or lipophilic carbon sources such as fats, oils, partial glycerides, fatty acids, fatty alcohols, long-chain saturated or unsaturated hydrocarbons.

In connection with the present invention, the term “aqueous medium” is understood as meaning a composition which comprises at least 5% by weight of water, based on the total composition under consideration.

Where average values are stated hereinbelow, then, unless stated otherwise, these are number-averaged average values.

Unless stated otherwise, percentages are data in per cent by weight.

Wherever measurement values are stated hereinbelow, then, unless stated otherwise, these have been determined at a temperature of 25° C. and a pressure of 1013 mbar.

The “pH” in connection with the present invention—unless stated otherwise—is defined as the value which is measured for the relevant composition at 25° C. after stirring for five minutes using a pH electrode calibrated in accordance with ISO 4319 (1977).

The composition according to the instant invention preferably is an aqueous composition.

A preferred composition according to the invention is characterized in that the pH of the mixture composition at 25° C. is from 3.0 to 9, preferably from 4.0 to 7 and particularly preferably from 5.0 to 6.6.

The composition according to the instant invention preferably comprises as component A) at least one biosurfactant selected from rhamnolipids, sophorolipids, glucose lipids, cellulose lipids, mannosylerythritol lipids and trehalose lipids, preferably rhamnolipids and sophorolipids. The biosurfactants, in particular glycolipid surfactants, can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789 or the documents cited therein. Suitable biosurfactants can be acquired e.g. from Soliance, France.

Preferably, the composition according to the instant invention has, as biosurfactant at least one selected from rhamnolipids, in particular mono-, di- or polyrhamnolipids, glucolipids, in particular mono-, di- or polyglucolipids, and sophorolipids, in particular mono-, di- or polysophorolipids, most preferably rhamnolipids.

The term “rhamnolipids” in the context of the present invention preferably is understood to mean particularly compounds of the general formula (II) and salts thereof,

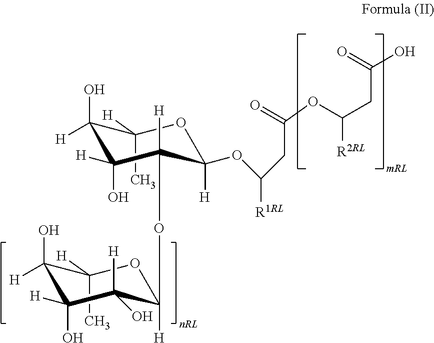

where
mRL=2, 1 or 0,
nRL=1 or 0,
R^{1RL} and R^{2RL}=mutually independently, identical or different, organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CH₃ where o=1 to 23, preferably 4 to 12. If nRL=1, the glycosidic bond between the two rhamnose units is preferably in the α-configuration. The optically active carbon atoms of the fatty acids are preferably present as R-enantiomers (e.g. (R)-3-{(R)-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (II) or salts thereof, where n=1.

The term “mono-rhamnolipid” in the context of the present invention is understood to mean compounds of the general formula (II) or salts thereof, where n=0.

Distinct rhamnolipids are abbreviated according to the following nomenclature:
“diRL-CXCY” are understood to mean di-rhamnolipids of the general formula (II), in which one of the residues R^{1RL} and R^{2RL}═(CH₂)ₒ—CH₃ where o=X−4 and the remaining residue R¹ or R²═(CH₂)ₒ—CH₃ where o=Y−4.
“monoRL-CXCY” are understood to mean mono-rhamnolipids of the general formula (II), in which one of the residues R^{1RL} and R^{2RL}═(CH₂)ₒ—CH₃ where o=X−4 and the remaining residue R^{1RL} or R^{2RL}═(CH₂)ₒ—CH₃ where o=Y−4.

The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “:Z”, this signifies that the respective residue R^{1RL} and/or R^{2RL} is equal to an unbranched, unsubstituted hydrocarbon residue having X−3 or Y−3 carbon atoms having Z double bonds.

Methods for preparing the relevant rhamnolipids are disclosed, for example, in EP2786743 and EP2787065.

Rhamnolipids applicable in the context of the instant invention can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been improved for higher rhamnolipid titres by genetical modification can also be used in the context of the instant invention; such cells have for example been disclosed by Lei et al. in Biotechnol Lett. 2020 June; 42(6):997-1002.

Rhamnolipids produced by Pseudomonas aeruginosa are commercially available from Jeneil Biotech Inc., e.g. under the tradename Zonix,from Logos Technologies (technology acquired by Stepan), e.g. under the tradename NatSurFac, from Biotensidion GmbH, e.g. under the tradename Rhapynal, from AGAE technologies, e.g. under the name R90, R95, R95Md, R95Dd, from Locus Bio-Energy Solutions and from Shanghai Yusheng Industry Co. Ltd., e.g. under the tradename Bio-201 Glycolipids.

The present invention provides a composition preferably comprising as biosurfactant rhamnolipids, characterized in that the biosurfactant component A) comprises
51% by weight to 95% by weight, preferably 70% by weight to 90% by weight, particularly preferably 75% by weight to 85% by weight, of diRL-C10C10 and
0.5% by weight to 9% by weight, preferably 0.5% by weight to 3% by weight, particularly preferably 0.5% by weight to 2% by weight, of monoRL-C10C10
where the percentages by weight refer to the sum of all of the rhamnolipids present.

A preferred composition according to the invention is characterized in that the composition comprises as biosurfactant rhamnolipids as described above with a content of
0.5% by weight to 15% by weight, preferably 3% by weight to 12% by weight, particularly preferably 5% by weight to 10% by weight, of diRL-C10C12:1,
where the percentages by weight refer to the sum of all of the rhamnolipids present.

A further preferred composition according to the invention is characterized in that the composition comprises as biosurfactant rhamnolipids as described above with a content of
0.5 to 25% by weight, preferably 5% by weight to 15% by weight, particularly preferably 7% by weight to 12% by weight, of diRL-C10C12,
where the percentages by weight refer to the sum of all of the rhamnolipids present.

It is also preferred if the composition according to the invention is characterized in that the composition comprises as biosurfactant rhamnolipids as described above with a content of
0.1% by weight to 5% by weight, preferably 0.5% by weight to 3% by weight, particularly preferably 0.5% by weight to 2% by weight, of monoRL-C10C12 and/or, preferably and
0.1% by weight to 5% by weight, preferably 0.5% by weight to 3% by weight, particularly preferably 0.5% by weight to 2% by weight, of monoRL-C10C12:1,
where the percentages by weight refer to the sum of all of the rhamnolipids present.

In the context of the present invention, the term “sophorolipids” preferably is understood as meaning compounds of the general formulae (IIIa) and (IIIb) and salts thereof

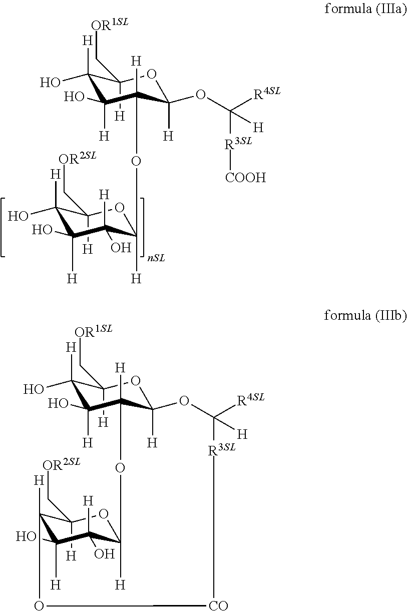

where
R^{1SL}═H or CO—CH₃,
R^{2SL}═H or CO—CH₃,
R^{3SL}=a divalent organic moiety which comprises 6 to 32 carbon atoms and which is unsubstituted or substituted by hydroxyl functions, is unbranched and optionally comprises one to three double or triple bonds,
R^{4SL}═H, CH₃ or a monovalent organic radical which comprises 2 to 10 carbon atoms and which is unsubstituted or substituted by hydroxyl functions, which is unbranched and which optionally comprises one to three double or triple bonds, and
nSL=1 or 0.

Sophorolipids may be used in accordance with the invention in their acid form or their lactone form. Preferred compositions according to the instant invention comprise a sophorolipid in which the ratio by weight of lactone form to acid form is in the range of 20:80 to 80:20, especially preferably in the ranges of 30:70 to 40:60.

To determine the content of sophorolipids in the acid or lactone form in a formulation, refer to EP1411111B1, page 8, paragraph [0053].

In connection with the present invention, the term “glucolipids” preferably is understood as meaning compounds of the general formula (IV) and salts thereof,

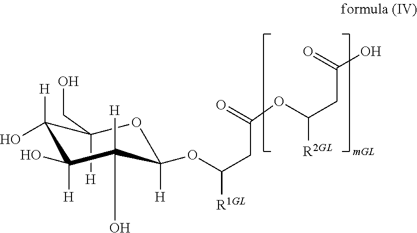

where
mGL=1 or 0,
R^{1GL} and R^{2GL}=independently of one another identical or different organic radical having 2 to 24 carbon atoms, in particular optionally branched, optionally substituted, in particular hydroxy-substituted, optionally unsaturated, in particular optionally mono-, di- or triunsaturated, alkyl radical, preferably one selected from the group consisting of pentenyl, heptenyl, nonenyl, undecenyl and tridecenyl and (CH₂)ₒ—CH₃ where o=1 to 23, preferably 4 to 12.

Distinct glucolipids are abbreviated according to the following nomenclature:
“GL-CXCY” is understood as meaning glucolipids of the general formula (IV) in which one of the radicals R^{1GL} and R^{2GL}═(CH₂)ₒ—CH₃ where o=X−4 and the remaining radical R^{1GL} or R^{2GL}═(CH₂)ₒ—CH₃ where o=Y−4.

The nomenclature used thus does not differentiate between “CXCY” and “CYCX”.

If one of the aforementioned indices X and/or Y is provided with “:Z”, then this means that the respective radical R^{1GL} and/or R^{2GL}=an unbranched, unsubstituted hydrocarbon radical with X−3 or Y−3 carbon atoms having Z double bonds.

Methods for production of glucolipids can be carried out as described in WO2019154970.

The present invention provides a composition preferably comprising as biosurfactant glucolipids, characterized in that the biosurfactant component A) comprises
1% by weight to 30% by weight, preferably 5% by weight to 25% by weight, particularly preferably 10% by weight to 20% by weight, of GL-C8C10,
where the percentages by weight refer to the sum of all of the glucolipids present.

A preferred composition according to the invention is characterized in that the composition comprises as biosurfactant glucolipids as described above with a content of
0.5% by weight to 20% by weight, preferably 3% by weight to 17% by weight, particularly preferably 5% by weight to 15% by weight, of GL-C10C12:1,
where the percentages by weight refer to the sum of all of the glucolipids present.

A further preferred composition according to the invention is characterized in that the composition comprises as biosurfactant glucolipids as described above with a content of
0.5% by weight to 20% by weight, preferably 2% by weight to 15% by weight, particularly preferably 3% by weight to 12% by weight, of GL-C10C12
where the percentages by weight refer to the sum of all of the glucolipids present.

A particularly preferred composition according to the invention is characterized in that the composition comprises as biosurfactant glucolipids as described above with a content of
1% by weight to 30% by weight, preferably 5% by weight to 25% by weight, particularly preferably 10% by weight to 20% by weight, of GL-C8C10,
0.5% by weight to 20% by weight, preferably 3% by weight to 17% by weight, particularly preferably 5% by weight to 15% by weight, of GL-C10C12:1,
0.5% by weight to 20% by weight, preferably 2% by weight to 15% by weight, particularly preferably 3% by weight to 12% by weight, of GL-C10C12
where the percentages by weight refer to the sum of all of the glucolipids present.

A very particularly preferred composition according to the invention is characterized in that the composition comprises as biosurfactant glucolipids as described above with a content of
10% by weight to 20% by weight, of GL-C8C10,
5% by weight to 15% by weight, of GL-C10C12:1,
3% by weight to 12% by weight, of GL-C10C12
where the percentages by weight refer to the sum of all of the glucolipids present.

A preferred composition according to the instant invention is characterized in that the biosurfactant is selected from rhamnolipids, sophorolipids and glucolipids.

The composition according to the instant invention comprises at least one sulfonic or sulfinic acid derivative of general formula (I); general formula (I) contains at least one chiral carbon atom. General formula (I) encompasses all possible enantiomers in context of the instant invention.

Preferably the composition according to the instant invention comprises at least one sulfonic or sulfinic acid derivative of general formula (I) in form of an enantiomer-mixture.

Preferably the composition according to the instant invention comprises at least one sulfonic or sulfinic acid derivative of general formula (I) selected from those with
n=2, R¹═H and R²═COOH,
n=1, R¹═H and R²═COOH,
with
n=2, R¹═H and R²═COOH being the most preferred,
or any salt thereof.

The components present in the composition according to the instant invention are present at least partially as salts on account of the given pH.

In preferred compositions according to the invention the cations of the salts of the sulfonic or sulfinic acid derivative of general formula (I) present are selected from the group comprising, preferably consisting of, Li⁺, Na⁺, K⁺, Mg²⁺, Ca²⁺, Al³⁺, Zn²⁺, NH₄⁺, primary ammonium ions, secondary ammonium ions, tertiary ammonium ions and quaternary ammonium ions, with Na⁺ and K⁺ being preferred, Na⁺ being most preferred.

A preferred composition according to the instant invention is characterized in that
component A) is comprised in an amount of 5% by weight to 70% by weight, preferably 6% by weight to 60% by weight, particularly preferably 10% by weight to 55% by weight and especially preferably 20% by weight to 50% by weight, and
component B) is comprised in an amount of 0.01% by weight to 10% by weight, preferably 0.1% by weight to 5% by weight, particularly preferably 0.2% by weight to 2% by weight,
where the percentages by weight refer to the total composition.

A preferred composition according to the instant invention is characterized in that the weight ratio of component A) to component B) is in the range of from 10:1 to 5000:1, preferably from 20:1 to 1000:1, more preferably from 25:1 to 250:1.

It is a further object of the instant invention to use at least one sulfonic or sulfinic acid derivative of general formula (I)

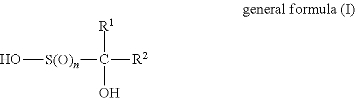

with
n=1 or 2,
R¹ selected from H and C₁-C₆ alkyl,
R² selected from COOH, SO₃H and CH(OH)SOₘ—OH with m=1 or 2, preferably m=n,
or any salt thereof for improving the odor of a composition comprising at least one biosurfactant.

All preferred uses according to the instant invention use the preferred sulfonic or sulfinic acid derivatives and biosurfactants as described above for the compositions according to the instant invention.

The compositions according to the invention can advantageously be incorporated into formulations, in particular in cosmetic formulations.

Consequently, a further subject matter of the present invention is the use of the compositions according to the invention for producing formulations, in particular cosmetic formulations, and also the formulations, in particular cosmetic formulations, which comprise the composition according to the invention.

The formulation according to the invention preferably contains 0,5% by weight to 20% by weight, preferably 2% by weight to 15% by weight, particularly preferably 3% by weight to 12% by weight, of biosurfactants, where the percentages by weight refer to the overall formulation.

Besides the compositions according to the invention, preferred formulations according to the invention comprise at least-next to the biosurfactant—one further surfactant, it being possible to use, for example, anionic, nonionic, cationic and/or amphoteric surfactants. Preferably, from an applications-related point of view, preference is given to mixtures of anionic and nonionic surfactants. The total surfactant content of the formulation is preferably 5 to 60% by weight and particularly preferably 15 to 40% by weight, based on the total formulation.

Furthermore, the formulations according to the invention can comprise at least one additional component selected from the group of
emollients,
emulsifiers,
thickeners/viscosity regulators/stabilizers,
UV photoprotective filters,
antioxidants,
hydrotropes (or polyols),
solids and fillers,
film formers,
pearlescent additives,
deodorant and antiperspirant active ingredients,
insect repellents,
self-tanning agents,
preservatives,
conditioners,
perfumes,
dyes,
odour absorbers,
cosmetic active ingredients,
care additives,
superfatting agents,
solvents.

Substances which can be used as exemplary representatives of the individual groups are known to the person skilled in the art and can be found for example in the German application DE 102008001788.4. This patent application is hereby incorporated by reference and thus forms part of the disclosure.

As regards further optional components and the amounts of these components used, reference is made expressly to the relevant handbooks known to the person skilled in the art, for example K. Schrader, “Grundlagen und Rezepturen der Kosmetika [Fundamentals and Formulations of Cosmetics]”, 2nd edition, page 329 to 341, Hüthig Buch Verlag Heidelberg.

The amounts of the respective additives are dependent on the intended use.

Typical guide formulations for the respective applications are known prior art and are contained for example in the brochures of the manufacturers of the respective base materials and active ingredients. These existing formulations can generally be adopted unchanged. If required, however, the desired modifications can be undertaken without complication by means of simple experiments for the purposes of adaptation and optimization.

The compositions according to the invention and the formulations according to the invention comprising the composition according to the invention can advantageously be used for the cleaning of surfaces. In this form of the use according to the invention, the surface is preferably the surface of a living being, in particular of a person, with such surfaces particularly preferably being selected from skin and hair. In the context of the inventive use on the surface of a living being, the inventive use is a non-therapeutic use, preferably a cosmetic use.

The examples adduced hereinafter describe the present invention by way of example, without any intention that the invention, the scope of application of which is apparent from the entirety of the description and the claims, be restricted to the embodiments specified in the examples.

### BRIEF DESCRIPTION OF THE FIGURES

FIG. 1 shows the discolouration over time for different rhamnolipid containing compositions.

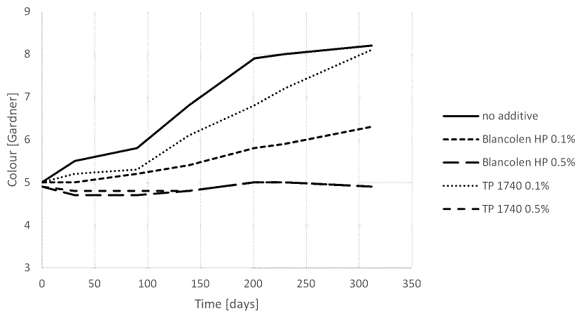

### EXAMPLES

Rhamnolipids were prepared as described in EP3023431 and analysed.

The proportion of rhamnolipids and salts thereof was >90% by weight based on the dry mass. The relative proportions of the various rhamnolipid congeners in per cent by weight of the sum total of all rhamnolipids are given in the following table. Here, the ratios refer to the acid form which is quantified in the HPLC analysis.

TABLE 1
Composition of the rhamnolipids used. Data in % by
weight of the respective congener (as acid form) based
on the sum total of all rhamnolipids (as acid form).

| | |
|---|---|
| diRL-C8C10 | 15.8 |
| diRL-C10C10 | 66.4 |
| diRL-C10C12:1 | 6.4 |
| diRL-C10C12 | 6.2 |
| monoRL-C10C10 | 2.4 |
| other rhamnolipids | 2.8 |

The protein content was determined by the photometric bicinchoninic assay (BCA assay, ThermaFisher Scientific) and was <1% by weight based on the dry mass of the rhamnolipid.

As sophorolipids REWOFERM® SL ONE (Evonik), an aqueous solution of sophorolipids in lactone-and acid form was used.

Glucolipids were prepared as described in example 2 of WO2019154970.

### Example 1: Odour Panel for Rhamnolipids Containing the Di-Sodium Salt of 2-hydroxysulfonatoacetate

Three samples were prepared by using rhamnolipid material with a rhamnolipid content of 47.4%. In the first sample no additive was added. In the second sample 0.5% of the di-sodium salt of 2-hydroxysulfonatoacetate (Blancolen H P, L. Brüggemann GmbH & CO KG), in a third sample 0.2% of the di-sodium salt of 2-hydroxysulfonatoacetate (Blancolen H P, L. Brüggemann GmbH & CO KG), was added. The samples had a pH of 5.6. The samples were stored in the suitable test bottles at ambient temperature.

To guarantee uniform surroundings, the members of the test panel carried out the test in an “odour-neutral” room of a central lab.

Each test person made the odour test and assigned points according to the below given ranking:

| | |
|---|---|
| Good sample | 1 Point |
| Medium sample | 2 Points |
| Bad sample | 3 Points |

The test result is the sum of all point divided by the number of test persons. Additionally, the test persons assigned a ranking for all three samples.

Points between 1-3

| | Additive | | | | | | | | | |
|---|---|---|---|---|---|---|---|---|---|---|
| | amount | test person | | | | | | | | Rank- |
| No. | [%] | 1 | 2 | 3 | 4 | 5 | 6 | Total | Result | ing |
| 1 | no | 2 | 2 | 2 | 3 | 2 | 1 | 12 | 2.00 | 3 |
| | additive | | | | | | | | | |
| 2 | 0.50% | 2 | 3 | 2 | 1 | 1 | 1 | 10 | 1.67 | 1 |
| 3 | 0.20% | 2 | 3 | 2 | 2 | 1 | 1 | 11 | 1.83 | 2 |

Ranking

| | Additive | | | | | | | | | |
|---|---|---|---|---|---|---|---|---|---|---|
| | amount | test person | | | | | | | | Rank- |
| No. | [%] | 1 | 2 | 3 | 4 | 5 | 6 | Total | Result | ing |
| 1 | no | 1 | 1 | 3 | 3 | 3 | 3 | 14 | 2.33 | 3 |
| | additive | | | | | | | | | |
| 2 | 0.50% | 2 | 2 | 1 | 1 | 1 | 1 | 8 | 1.33 | 1 |
| 3 | 0.20% | 3 | 2 | 1 | 2 | 2 | 1 | 11 | 1.83 | 2 |

### Example 2: Odour Panel for Rhamnolipids Containing the Zinc Salt of 2-hydroxysulfonatoacetate

Three samples were prepared by using rhamnolipid material with a rhamnolipid content of 47.4%. In the first sample no additive was added. In the second sample 0.5% of the zinc salt of 2-hydroxysulfonatoacetate (TP1740, L. Brüggemann GmbH & CO KG), in a third sample 0.2% of the zinc salt of 2-hydroxysulfonatoacetate (TP 1740, L. Brüggemann GmbH & CO KG) was added. The samples had a pH of 5.8. The samples were stored in the suitable test bottles at ambient temperature.

To guarantee uniform surroundings, the members of the test panel carried out the test in an “odour-neutral” room of a central lab.

Each test person made the odour test and assigned points according to the below given ranking:

| | |
|---|---|
| Good sample | 1 Point |
| Medium sample | 2 Points |
| Bad sample | 3 Points |

The test result is the sum of all point divided by the number of test persons. Additionally, the test persons assigned a ranking for all three samples.

Points between 1-3

| | Additive | | | | | | | | | |
|---|---|---|---|---|---|---|---|---|---|---|
| | amount | test person | | | | | | | | Rank- |
| No. | [%] | 1 | 2 | 3 | 4 | 5 | 6 | Total | Result | ing |
| 1 | No | 3 | 3 | 2 | 1 | 2 | 2 | 13 | 2.17 | 3 |
| | additive | | | | | | | | | |
| 2 | 0.50% | 1 | 2 | 3 | 1 | 1 | 1 | 9 | 1.50 | 1 |
| 3 | 0.20% | 2 | 3 | 2 | 1 | 1 | 3 | 12 | 2.00 | 2 |

Ranking

| | Additive | | | | | | | | | |
|---|---|---|---|---|---|---|---|---|---|---|
| | amount | test person | | | | | | | | Rank- |
| No. | [%] | 1 | 2 | 3 | 4 | 5 | 6 | Total | Result | ing |
| 1 | No | 2 | 2 | 2 | 2 | 3 | 2 | 13 | 2.20 | 3 |
| | additive | | | | | | | | | |
| 2 | 0.50% | 1 | 2 | 3 | 1 | 1 | 1 | 9 | 1.60 | 1 |
| 3 | 0.20% | 3 | 1 | 1 | 3 | 1 | 3 | 12 | 1.80 | 2 |

### Example 3: Odour Panel for Sophorolipids Containing the Disodium Salt of 2-hydroxysulfonatoacetate or the Mixture of the Zinc Salts of 2-hydroxysulfinatoacetate, 2-hydroxysulfonatoacetate and Sulfite

Three samples were prepared by using sophorolipid material with a sophorolipid content of 46.4%. In the first sample a mixture of 0.5% of the zinc salts of 2-hydroxysulfinatoacetate, 2-hydroxysulfonatoacetate and sulfite (TP1647, L. Brüggemann GmbH & CO KG) was added. In a second sample no additive was used. In a third sample 0.5% of the disodium salt of 2-hydroxysulfonatoacetate (Blancolen H P, L. Brüggemann GmbH & CO KG) was added. The samples had a pH of 5.5. The samples were stored in the suitable test bottles at ambient temperature.

To guarantee uniform surroundings, the members of the test panel carried out the test in an “odour-neutral” room of a central lab.

Each test person made the odour test and assigned points according to the below given ranking:
Good sample 1 Point
Medium sample 2 Points
Bad sample 3 Points

The test result is the sum of all point divided by the number of test persons. Additionally, the test persons assigned a ranking for all three samples.

Points between 1-3

| | | test person | | | | | | | | |
|---|---|---|---|---|---|---|---|---|---|---|
| No. | Additive | 1 | 2 | 3 | 4 | 5 | 6 | Total | Result | Ranking |
| 1 | 0.5% TP 1647 | 2 | 1 | 2 | 3 | 1 | 2 | 11 | 1.83 | 2 |
| 2 | No additive | 2 | 1 | 3 | 2 | 2 | 3 | 13 | 2.17 | 3 |
| 3 | 0.5% Blancolen HP | 2 | 1 | 2 | 2 | 1 | 2 | 10 | 1.67 | 1 |

| Ranking | | test person | | | | | | | | |
|---|---|---|---|---|---|---|---|---|---|---|
| No. | Additive amount [%] | 1 | 2 | 3 | 4 | 5 | 6 | Total | Result | Ranking |
| 1 | 0.5% TP 1647 | 1 | 1 | 2 | 3 | 1 | 3 | 11 | 1.83 | 2 |
| 2 | No additive | 2 | 3 | 3 | 2 | 3 | 2 | 15 | 2.50 | 3 |
| 3 | 0.5% Blancolen HP | 3 | 2 | 1 | 1 | 1 | 1 | 9 | 1.50 | 1 |

### Example 4: Colour Preservation

A 47.4% aqueous rhamnolipid solution was used. The solution was stabilized with 12 ppm CIT/MIT (chloromethylisothiazolinone/methylisothiazolinone) against microbial growth and had a pH at 5.5. This solution was mixed with the di-sodium salt of 2-hydroxysulfonatoacetate (Blancolen H P, L. Brüggemann GmbH & CO KG) and the zinc salt of 2-hydroxysulfonatoacetate (TP1740, L. Brüggemann GmbH & CO KG) at concentrations of 0.1% and 0.5%. After the addition of the additives the pH was set to 5.6 with KOH. The mixture was incubated at 40° C. under permanent stirring. In regular intervals the samples were analyzed on Gardner colour number up to 312 days.

| | | additive | Colour [Gardner] | | | | | | |
|---|---|---|---|---|---|---|---|---|---|
| Sample | | amount | 0 | 31 | 90 | 139 | 201 | 230 | 312 |
| no. | Additive | [%] | days | days | days | days | days | days | days |
| 1 | — | — | 5 | 5.5 | 5.8 | 6.8 | 7.9 | 8 | 8.2 |
| 2 | Blancolen HP | 0.1% | 5 | 5 | 5.2 | 5.4 | 5.8 | 5.9 | 6.3 |
| 3 | Blancolen HP | 0.5% | 4.9 | 4.7 | 4.7 | 4.8 | 5 | 5 | 4.9 |
| 4 | TP1740 | 0.1% | 5 | 5.2 | 5.3 | 6.1 | 6.8 | 7.2 | 8.1 |
| 5 | TP1740 | 0.5% | 4.9 | 4.8 | 4.8 | 4.8 | 5 | 5 | 4.9 |

The experiment is repeated in the same way, however the pH is adjusted to 6.6 and analogue results are obtained.

### Example 5: Odour Panel for Glucolipids Containing the Disodium Salt of 2-hydroxysulfonatoacetate

Three samples were prepared by using glucolipid material with a content of 49.2%. In the first sample no additive was used. In a second sample 2.0% of the disodium salt of 2-hydroxysulfonatoacetate (Blancolen H P, L. Brüggemann GmbH & CO KG) was added. In a third sample 0.5% of the disodium salt of 2-hydroxysulfonatoacetate (Blancolen H P, L. Brüggemann GmbH & CO KG) was used. The samples had a pH of 6.0. The samples were stored in the suitable test bottles at ambient temperature.

To guarantee uniform surroundings, the test was carried out in an “odour-neutral” room of a central lab.

Each test person made the odour test and assigned points according to the below given ranking:
Good sample 1 Point
Medium sample 2 Points
Bad sample 3 Points

The test result is the sum of all point divided by the number of test persons. Additionally, the test persons assigned a ranking for all three samples.

Points between 1-3

| | | test person | | | | | | | | |
|---|---|---|---|---|---|---|---|---|---|---|
| No. | Additive | 1 | 2 | 3 | 4 | 5 | 6 | Total | Result | Ranking |
| 1 | No additive | 3 | 3 | 1 | 2 | 3 | 2 | 14 | 2.33 | 3 |
| 2 | 2.0% Blancolen HP | 2 | 1 | 2 | 1 | 1 | 2 | 9 | 1.50 | 1 |
| 3 | 0.5% Blancolen HP | 2 | 1 | 3 | 1 | 2 | 2 | 11 | 1.83 | 2 |

Ranking

| | | test person | | | | | | | | |
|---|---|---|---|---|---|---|---|---|---|---|
| No. | Additive amount [%] | 1 | 2 | 3 | 4 | 5 | 6 | Total | Result | Ranking |
| 1 | No additive | 3 | 3 | 1 | 3 | 3 | 2 | 15 | 2.50 | 3 |
| 2 | 2.0% Blancolen HP | 1 | 2 | 2 | 1 | 1 | 2 | 9 | 1.50 | 1 |
| 3 | 0.5% Blancolen HP | 2 | 1 | 3 | 2 | 2 | 2 | 12 | 2.00 | 2 |

### Example 6: Odour Panel for Glucolipids Containing the Disodium Salt of 2-hydroxysulfonatoacetate, the Zinc Salt of 2-hydroxysulfonatoacetate or the Zinc Salts of 2-hydroxysulfinatoacetate, 2-hydroxysulfonatoacetate and Sulfite at Higher Concentrations

Four samples were prepared by using glucolipid material with a content of 49.2%. In the first sample 5.0% of the disodium salt of 2-hydroxysulfonatoacetate (Blancolen H P, L. Brüggemann GmbH & CO KG) was added. In the second sample 5.0% of the zinc salts of 2-hydroxysulfinatoacetate, 2-hydroxysulfonatoacetate and sulfite (TP1647, L. Brüggemann GmbH & CO KG) was used. In a third sample no additive was added. In the fourth sample 5.0% of the zinc salt of 2-hydroxysulfonatoacetate (TP1740, L. Brüggemann GmbH & CO KG) was used. The samples had a pH of 8.0. The samples were stored in the suitable test bottles at ambient temperature.

To guarantee uniform surroundings, the test was carried out in an “odour-neutral” room of a central lab.

Each test person made the odour test and assigned points according to the below given ranking:

| | |
|---|---|
| Good sample | 1 Point |
| Medium sample | 2 Points |
| Bad sample | 3 Points |

The test result is the sum of all point divided by the number of test persons. Additionally, the test persons assigned a ranking for all three samples.

Points between 1-3

| | | test person | | | | | | | | |
|---|---|---|---|---|---|---|---|---|---|---|
| No. | Additive | 1 | 2 | 3 | 4 | 5 | 6 | Total | Result | Ranking |
| 1 | 5.0% Blancolen HP | 2 | 1 | 1 | 2 | 1 | 1 | 8 | 1.33 | 1 |
| 2 | 5.0% TP1647 | 3 | 2 | 3 | 2 | 2 | 1 | 13 | 2.17 | 2 |
| 3 | No additive | 3 | 2 | 3 | 1 | 3 | 2 | 14 | 2.33 | 3 |
| 4 | 5.0% TP1740 | 1 | 1 | 2 | 2 | 1 | 1 | 8 | 1.33 | 1 |

| | Additive | test person | | | | | | | | |
|---|---|---|---|---|---|---|---|---|---|---|
| No. | amount [%] | 1 | 2 | 3 | 4 | 5 | 6 | Total | Result | Ranking |
| 1 | 5.0% Blancolen HP | 2 | 2 | 1 | 3 | 1 | 2 | 11 | 1.83 | 1 |
| 2 | 5.0% TP1647 | 3 | 4 | 3 | 4 | 3 | 1 | 18 | 3.00 | 2 |
| 3 | No additive | 4 | 3 | 4 | 1 | 4 | 4 | 20 | 3.33 | 3 |
| 4 | 5.0% TP1740 | 1 | 1 | 2 | 2 | 2 | 3 | 11 | 1.83 | 1 |

1. A composition, comprising:
A) at least one biosurfactant;
B) at least one sulfonic or sulfinic acid derivative of formula (I) 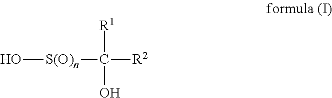
wherein
n=1 or 2,
R¹ is selected from the group consisting of H and C₁-C₆ alkyl, and
R² is selected from the group consisting of COOH, SO₃H and
CH(OH)SOₘ—OH with m=1 or 2,
or any salt thereof.

2. The composition according to claim 1, wherein the biosurfactant is selected from the group consisting of rhamnolipids, sophorolipids, and glucolipids.

3. The composition according to claim 1, wherein the composition comprises at least one sulfonic or sulfinic acid derivative of formula (I) selected from those with
n=2, R¹═H, and R²═COOH,
n=1, R¹═H, and R²═COOH;
or any salt thereof.

4. The composition according to claim 1, wherein in component A) the cations of the salts of the at least one sulfonic or sulfinic acid derivative of formula (I) present are selected from the group consisting of Li⁺, Na⁺, K⁺, Mg²⁺, Ca²⁺, Al³⁺, Zn²⁺, NH₄⁺, primary ammonium ions, secondary ammonium ions, tertiary ammonium ions, and, quaternary ammonium ions.

5. The composition according to claim 1, wherein
component A) is comprised in said composition in an amount of 5% by weight to 70% by weight, and
component B) is comprised in said composition in an amount of 0.01% by weight to 10% by weight,
wherein the percentages by weight refer to the total composition.

6. The composition according to claim 1, wherein a weight ratio of component A) to component B) is in a range of from 10:1 to 5000:1.

7. A method for improving an odor of a composition, the method comprising:
adding at least one sulfonic or sulfinic acid derivative of formula (I) 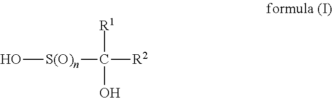
wherein
n=1 or 2,
R¹ is selected from the group consisting of H and C₁-C₆ alkyl, and
R² is selected from the group consisting of COOH, SO₃H and
CH(OH)SOₘ—OH with m=1 or 2,
or any salt thereof, to the composition, thereby improving the odor of the composition;
wherein the composition comprises at least one biosurfactant.

8. A method for producing a formulation, the method comprising:
adding the composition according to claim 1 to a surfactant to obtain the formulation.

9. A formulation, comprising: the composition according to claim 1.

10. The formulation according to claim 9, comprising at least one further surfactant.

11. The formulation according to claim 9, wherein a total surfactant content of the formulation is 5 to 60% by weight based on the total formulation.

12. A method for cleaning a surface, the method comprising:
applying the formulation according to claim 9 to the surface.

13. The composition according to claim 1, wherein m=n.

14. The composition according to claim 4, wherein the cation is Na⁺.

15. The composition according to claim 5, wherein component A) is comprised in the composition in an amount of 20% by weight to 50% by weight.

16. The composition according to claim 6, wherein the weight ratio of component A) to component B) is in a range of 25:1 to 250:1.

17. The composition according to claim 5, wherein component B) is comprised in the composition in an amount of 0.2% by weight to 2% by weight.

18. The method according to claim 8, wherein the formulation is a cosmetic formulation.

19. The formulation according to claim 10, wherein the at least one further surfactant is selected from the group consisting of anionic, nonionic, cationic and amphoteric surfactants.
